(19) United States
(12) Patent Application Publication
(10) Pub. No.: US 2015/0297731 A1
(43) Pub. Date: Oct. 22, 2015

(54) THERMOSENSITIVE INJECTABLE GLAUCOMA DRUG CARRIER GEL AND THE FABRICATING METHOD THEREOF

(71) Applicant: National Yang-Ming University, Taipei City (TW)

(72) Inventors: Shih-Hwa Chiou, Taipei City (TW); Jui-Ling Liu, Taipei City (TW); Dean-Mo Liu, Hsinchu City (TW)

(73) Assignee: National Yang-Ming University, Taipei City (TW)

(21) Appl. No.: 14/257,987

(22) Filed: Apr. 21, 2014

(51) Int. Cl. A61K 47/36; A61K 47/10; A61K 31/5377; A61K 47/18; A61K 47/24; A61K 31/5575; A61K 9/00; A61K 47/20

(52) U.S. Cl. CPC A61K 47/36; A61K 9/0019; A61K 47/10; A61K 47/20; A61K 47/186; A61K 47/24; A61K 31/5575; A61K 31/5377

(57) ABSTRACT

The present invention relates to a thermosensitive injectable glaucoma drug carrier gel and the fabricating method thereof. The thermosensitive injectable glaucoma drug carrier gel comprises: a polymer substrate comprising chitosan with hydrophilic and hydrophobic modification; an additive dispersed in the substrate, wherein the additive comprises a water/fat soluble glaucoma drug, a preservative, a solvent selected from glycerol, dimethyl sulfoxide (DMSO), ethanol, glycol or any combination thereof, and a basic structural stabilizer; and water.

### BACKGROUND OF THE INVENTION

1. Field of the Invention

The present invention relates to a glaucoma drug carrier gel comprising amphiphilic chitosan, which can be implanted into tissues for a long-term delivery of the glaucoma drug.

2. Description of Prior Art

Glaucoma eye drops, topical drop application of glaucoma drug to the eye's surface, is one of the most common conventional pharmaceutical (dosage) forms of glaucoma drugs. There are two problems associated with glaucoma eye drops: a substantial portion of the drop is lost due to overflow and poor patient compliance. Holden et al. discloses a hydrogel encapsulating glaucoma drugs, which is made from polyamidoamine (PAMAM) dendrimer and polyethylene glycol (PEG) rather than chitin (chitosan). The hydrogel disclosed by Holden et al. is not injectable and difficult to be implanted into animal eyes. (Christopher A. Holden, Puneet Tyagi, Ashish Thakur, Rajendra Kadam, Gajanan Jadhav, Uday B. Kompella, Hu Yang, “Polyamidoamine dendrimer hydrogel for enhanced delivery of antiglaucoma drugs”, Nanomedicine: Nanotechnology, Biology, and Medicine, 2012, 8, 776-783). Prior arts have disclosed injectable hydrogels for the treatment of glaucoma, but the hydrogels are not used as encapsulating material for drug delivery and the treatment mechanisms are different from the present invention.

FR2909285A1 patent publication relates to an injectable hydrogel carrying anti-cell adhesion and anti-fibrosis drugs for the treatment of glaucoma and healing of ophthalmic surgical wounds. US2005277864A1 relates to a method of using an injectable hydrogel implant for glaucoma treatment. These two patent publications do not disclose any methods of using injectable hydrogels to deliver drugs for the treatment of glaucoma. TW Patent I386224 discloses a method of using modified chitosan as an injectable gel but fails to provide a method of treating glaucoma (such as injection methods, injection sites, dosages, etc.). In addition, drug molecules are encapsulated in a magnetic-sensitive nanocapsule and the rupture of which is controlled by an external magnetic field. When the capsule is ruptured, drug molecules are released to the injectable gel which in turn slowly releases them in situ. A carrier gel which wraps drug molecules by mixing chitosan solution with the drug molecules is not provided by this patent.

### SUMMARY OF THE INVENTION

The present invention provides a method for manufacturing a thermosensitive injectable glaucoma drug carrier gel, comprising the steps of: providing 0.1-10% (w/v) amphiphilically modified chitosan solution; and at 4-20° C. adding 50-100 μg/ml water/fat soluble glaucoma drugs, 0.001-0.02% (w/v) preservatives, 5-20% (v/v) solvent selected from glycerol, dimethyl sulfoxide (DMSO), ethanol or ethylene glycol, or any combination thereof, and 10-50% (w/v) basic structural stabilizer to form a chitosan sol having the drug encapsulated therein, wherein the chitosan sol forms a solid gel when the temperature is increased to 30-40° C. It also provides a thermosensitive injectable glaucoma drug carrier gel, comprising: a polymer matrix comprising amphiphilically modified chitosan; an additive dispersed in the matrix, wherein the additive contains a water/fat soluble glaucoma drug, a preservative, a solvent selected from glycerol, dimethyl sulfoxide (DMSO), ethanol or ethylene glycol, or any combination thereof, and a basic structural stabilizer; and water.

### BRIEF DESCRIPTION OF THE DRAWINGS

FIG. 1 shows the transition of the modified chitin gel from liquid form to gel form before and after the temperature was changed from low temperature to 37° C.

FIG. 2 shows a curve of viscosity changes of the gel, with or without the preservative benzalkonium chloride, detected at various frequencies by a rheometer.

FIG. 3 shows the gel added with 0.01% and 002% of benzalkonium chloride, respectively, from Day 1 to Day 7 at 4° C. and 25° C., respectively. No dehydration was observed.

FIG. 4 (A) shows the results of in vitro drug delivery by the gel with or without the preservative benzalkonium chloride being added. The gel with the preservative delivered the drug faster than the one without the preservative. FIG. 4 (B) shows the results of in vitro drug delivery by the gel at various temperatures. The higher the temperature was the faster the drug was delivered by the gel.

FIG. 5 shows the results of in vitro drug delivery by the gel before and after the gel was radiated by γ-rays. After the gel was being radiated by γ-rays, the amount of drug delivered increased significantly at the first 7 hours.

### DETAILED DESCRIPTION OF THE INVENTION

The present invention provides a chitosan hydrogel having both hydrophilic and hydrophobic properties, which is not only injectable but is also capable of wrapping water/fat soluble glaucoma drugs in order to be implanted into eye tissues for long-term controlled drug delivery. The primary purpose of the present invention is to disclose a thermosensitive injectable glaucoma drug carrier gel which can be injected into body tissues to fix water/fat soluble glaucoma drugs in the eye tissues. After being fixed, the glaucoma drug carried by the carrier gel can be delivered slowly as a long-acting drug. The present invention solves the problems associated with conventional eye drops, namely, frequent topical applications or forgotten applications.

In addition, the thermosensitive injectable gel is rich in water, highly bio-compatible, highly bio-degradable and non-toxic, the cut of the implant is small and the implant can be removed without going through a surgery. The present invention provides a highly potential pharmaceutical formulation for the treatment of glaucoma because of it low cost, uncomplicated processes and production lines.

Unless defined otherwise, all technical and scientific terms used herein have the same meaning as commonly understood to one of ordinary skill in the art to which the present invention belongs. The meaning and scope of these terms should be clear; however, in the case of any potential ambiguity, definitions provided herein supersede any dictionary or extrinsic definition.

The singular forms “a,” “an,” and “the” include the plural forms and vice versa unless the context clearly dictates otherwise.

The term “strain” used herein refers to the ratio of horizontal displacement and height resulted from a force (F) imposed on the gel of the present invention. It is the deformation rate of an object resulted from a force imposed onto the object. The deformation rate is generally presented as %.

The present invention provides a method for manufacturing a thermosensitive injectable glaucoma drug carrier gel, comprising the steps of: providing 0.1-10% (w/v) amphiphilically modified chitosan solution; and at 4-20° C. adding 50-100 μg/ml water/fat soluble glaucoma drugs, 0.001-0.02% (w/v) preservatives, 5-20% (v/v) solvent selected from glycerol, dimethyl sulfoxide (DMSO), ethanol or ethylene glycol, or any combination thereof, and 10-50% (w/v) basic structural stabilizer to form a chitosan sol having the drug encapsulated therein, wherein the chitosan sol forms a solid gel when the temperature is increased to 30-40° C.

In a preferred embodiment, the concentration of the amphiphilically modified chitosan solution is 0.1-3% (w/v); the chitosan solution is prepared from 95% deacetylated chitosan powder having a molecular weight of 50 kDa˜250 kDa. Preferably, the chitosan solution is hydrophilically modified by haloacetic acid, and the haloacetic acid is chloroacetic acid, dichloroacetic acid, trichloroacetic acid, bromoacetic acid, dibromoacetic acid or bromochloroacetic acid. Preferably, the chitosan is hydrophobically modified by 2-12 carbons long-chain anhydride, and the anhydride is acetic anhydride or hexanoyl anhydride.

In a preferred embodiment, the glaucoma drug is Latanoprost or Timolol maleate. In another preferred embodiment, the preservative is benzalkonium chloride. In a preferred embodiment, the basic structure stabilizer is sodium β-glycerophosphate, genipin, sodium bicarbonate, or any combination thereof. In another preferred embodiment, after the step of forming chitosan gel, the present invention further comprises a step of radiating γ-rays at the gel at a dose of 3-10 KGy.

The present invention provides a thermosensitive injectable glaucoma drug carrier gel, comprising: a polymer matrix comprising amphiphilically modified chitosan; an additive dispersed in the matrix, wherein the additive contains a water/fat soluble glaucoma drug, a preservative, a solvent selected from glycerol, dimethyl sulfoxide (DMSO), ethanol or ethylene glycol, or any combination thereof, and a basic structural stabilizer; and water.

In a preferred embodiment, the thermosensitive injectable glaucoma drug carrier gel is prepared by aforementioned method. Preferably, the drug carrier gel does not contain magnetic-sensitive nanocapsule and can be prepared in jelly form or toothpaste form.

### EXAMPLES

The present invention can be embodied by a plurality of examples and it is not limited to the following examples. The examples below are non-limiting and are merely representative of various aspects and features of the present invention.

### Example 1

### Preparation of the Carrier Gel Encapsulating Glaucoma Drug

First, the amphiphilic chitosan solution at a concentration of 01%-10% (w/v) (preferably 0.1%-3% (w/v)) was prepared. In the present invention, the chitosan solution was prepared by 95% deacetylation of chitosan powder having a molecular weight of 50 kDa-250 kDa. The chitosan solution was first hydrophilically modified by haloacetic acid and then hydrophobically modified by 2-12 carbons long-chain anhydrides. The haloacetic acid was chloroacetic acid, dichloroacetic acid, trichloroacetic acid, bromoacetic acid, dibromoacetic acid or bromochloroacetic acid; the anhydride was acetic anhydride or hexanoyl anhydride. The modified chitosan had negative charge zeta potential, bio-degradable property and was capable of self-assembling into micelles. The solvent of the chitosan solution comprised of water or a mixture of water and oil, i.e., 1-20% organic solvent was added into 80-99.5% (w/v) diluted solution. The oil could be dimethyl sulfoxide (DMSO), ethanol, glycol or glycerol. For example, the added amount of glycerol was 5-20% (v/v) and the added amount of DMSO was 0.001-0.1% (v/v). Solutes and solvents were mixed by using electric rotary blender and magnetic blender.

The steps for synthesizing amphiphilic chitosan (CHC) powder were reported as the following:

1. 20 g chitosan was placed in a three-neck round-bottom flask, 200 ml isopropanol was added into the flask, stirred for 30 minutes to form a suspension.

2. 5 ml, 13.3N sodium hydroxide solution was added every 5 minutes for a total of ten times and the sum total was 50 ml.

3. Stirred for 30 minutes, 100 g chloroacetic acid was added in 5 equal parts in 5 minutes. Chloroacetic acid was added slowly to assure that it was fully dissolved.

4. The solution was heated in an oil bath to 60° C., reacted for 4 hours. The product was collected by suction filtration and concurrently washed with water: methanol solution (v/v 1:9).

5. The product was subsequently dried in an oven at 60° C. for 1 day to yield a white to light yellow N, O-carboxymethyl chitosan (NOCC) powder which was water soluble.

6. 4 g NOCC was placed in a 250 ml reaction flask, 100 ml pure water was added, stirred thoroughly for 1 day to make sure NOCC was fully dissolved in the water.

7. 50 ml methanol was added and mixed thoroughly. 2.8 ml hexanoyl anhydride was added and the mixture was allowed to react for 24 hours.

8. After the mixture was thoroughly reacted, the solution was collected by dialysis bag. It was dialyzed by water and ethanol (1:4) for 1 day, and then was dialyzed by ethanol for 2 days to remove acids and ions.

9. The collected product was dried at 60° C. for 1 day to yield amphiphilic chitosan powder.

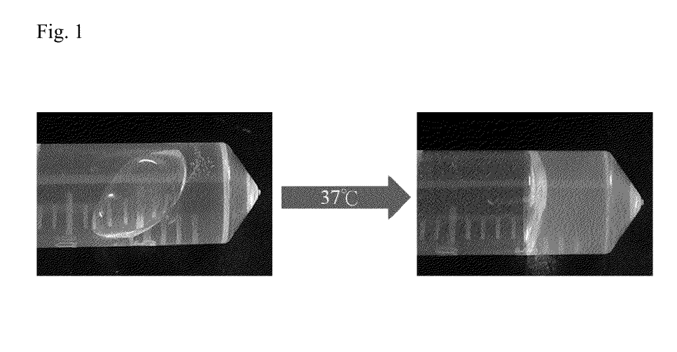

Taking advantage of the negative charge zeta potential and the capability of self-assembling into micelles of the modified amphiphilic chitosan (chitin), water/fat soluble glaucoma drugs (for example Latanoprost (50-1000 μg/ml), timolol maleate), preservatives (Benzalkonium chloride, 0.001-0.02% (w/v)) and other molecules to be carried were added into 0.1-10% (w/v) (preferably 0.1-3% (w/v)) modified chitosan solution at 4-20° C. for encapsulating. In order to stabilize the structure of the carrier gel, glycerol having multiple hydroxyl groups (—OH) was added to form hydrogen bonds with chitosan. Sodium β-glycerophosphate (10-50% (w/v)) was added as cross-linking agent (basic structure stabilizer). After the amphiphilic chitosan was placed at 37° C. the carrier gel transformed into a non-fluid gel, as shown in FIG. 1. The gel prepared in accordance with aforementioned method was in liquid form when it was placed at a temperature lower than 20° C., it became non-fluid gel when the temperature was increased to 30° C. The preservative added to the gel was used primarily to extend the shelf-life of the gel and glaucoma drugs wrapped by the gel. Benzalkonium chloride was one of the most common preservatives used in eye drops for purposes of disinfection and sterilization. It was not a strong agent for disinfection, but inexpensive, low in toxicity and not irritating. Furthermore, additional ingredients such as polymer electrolytes and cross-linking agents, sodium alginate and genipin, could also be added to modify the properties of the carrier. Alternatively, by adjusting the ratio of additives the physical properties of hardness and fluidity could also be modified.

Physical Properties of the Amphiphilic Chitosan Gel

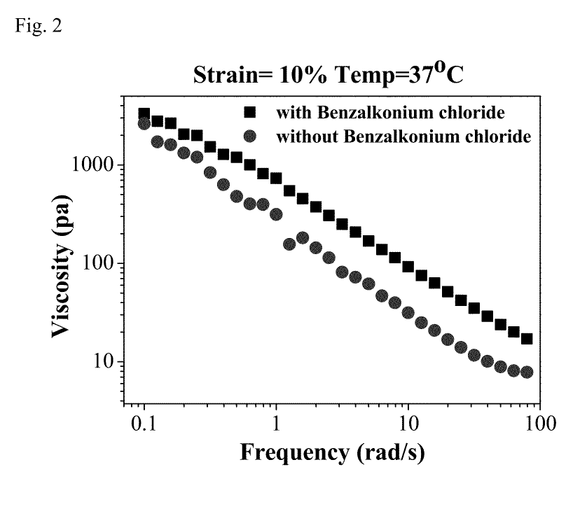

The present invention also examined the fluidity and deformation of the amphiphilic chitosan gel. These physical properties related to the gel's viscosity, elasticity and structural strength. As shown in FIG. 2, the physical properties of the gel were analyzed by studying the viscosity and angular frequency of the gel at various applied shear stresses using a rheometer. As it showed, the amount of deformation (strain) of the gel was 10% of the gel's thickness.

Stability of the Gel

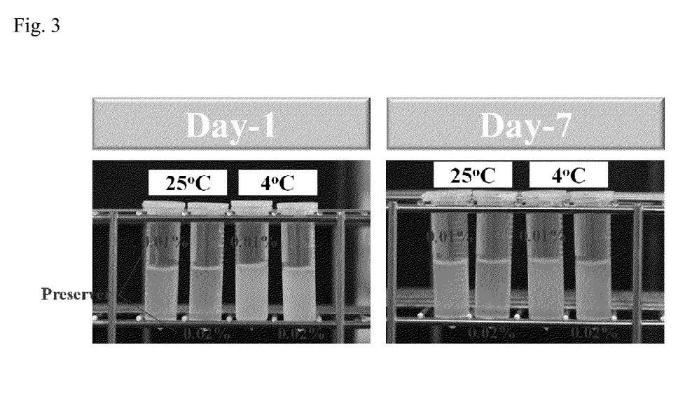

The present invention also examined the preservation and stability of the gel after benzalkonium chloride was being added into the gel. Dehydration might occur since benzalkonium chloride was a salt. After the gel was formed it was observed at 4° C. and 25° C. from Day 1 to Day 7 and the observations were shown in FIG. 3. No dehydration was observed from Day 1 to Day 7.

γ-Ray Radiation for Sterilization and Disinfection

Since the gel was designed to be injected into eye tissues or other tissues of human bodies, the gel was radiated by γ-ray (at a dose of 3-10 kGy) for sterilization and disinfection to enhance its safety, to minimize patients' risks and to meet biomedical and pharmaceutical standards and guidelines.

Glaucoma Drug Delivery

Since the gel was designed to be implanted into eye tissues, and the encapsulated drug was to be delivered as a long-term released drug for the treatment of glaucoma, in vitro drug delivery, simulating drug delivery in the conditions of animal bodies, was studied. 0.5 ml gel was placed in a 1.5 ml centrifuge tube, 1 ml phosphate buffer solution (pH 7.4) similar to human body fluid was added to the tube, phosphate buffer solutions were refreshed at predetermined time and the replaced phosphate buffer solutions containing glaucoma drug were quantitatively analyzed by high performance liquid chromatography (HPLC). Fat soluble glaucoma drug Latanoprost was used as the model drug to study drug delivery.

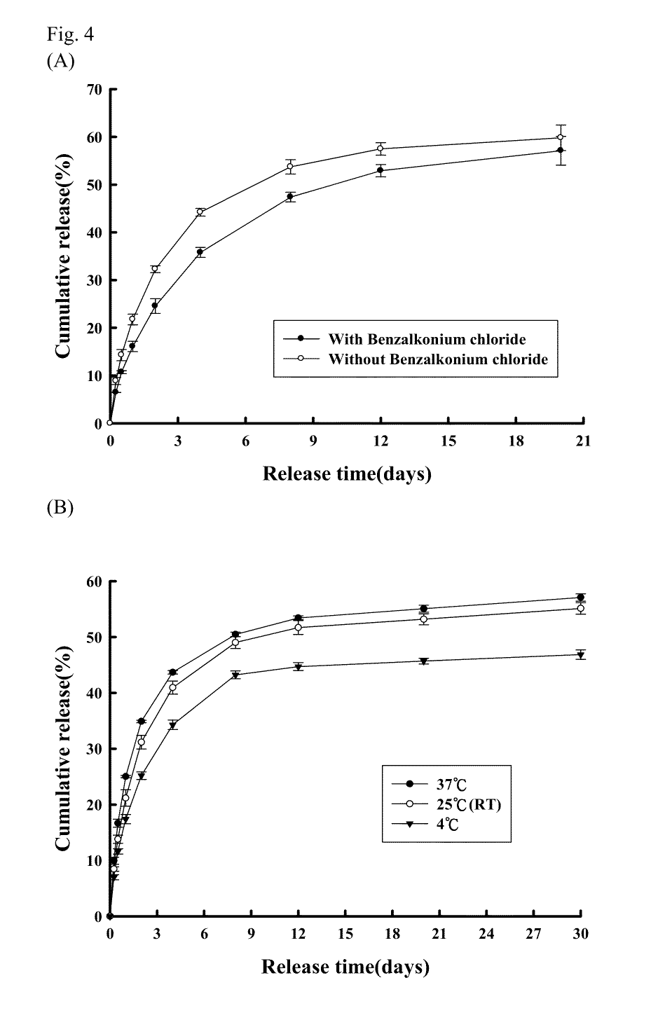

Additives in the gel might affect the composition and structure of the gel, which might further affect drug delivery. As shown in FIG. 4(A), benzalkonium chloride, added in the gel, affected drug delivery. Since the gel is thermo-sensitive and the injected gel are to be exposed to various temperatures, drug deliveries at various temperatures were studied and the results were shown in FIG. 4(B).

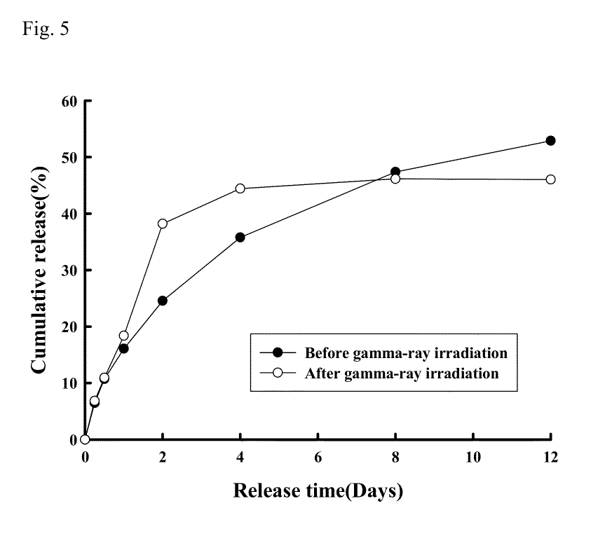

Since γ-rays, high-energy electromagnetic radiation rays, may cause harms and damages to the gel, which may further affect drug delivery, drug delivery after the gel was being radiated by γ-rays was studied. The results were shown in FIG. 5.

One skilled in the art readily appreciates that the present invention is well adapted to carry out the objects and obtain the ends and advantages mentioned, as well as those inherent therein. The gels and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for manufacturing a thermosensitive injectable glaucoma drug carrier gel, comprising the steps of: providing 0.1-10% (w/v) amphiphilically modified chitosan solution; and at 4-20° C. adding 50-100 μg/ml water/fat soluble glaucoma drugs, 0.001-0.02% (w/v) preservatives, 5-20% (v/v) solvent selected from glycerol, dimethyl sulfoxide (DMSO), ethanol or ethylene glycol, or any combination thereof, and 10-50% (w/v) basic structural stabilizer to form a chitosan sol having the drug encapsulated therein, wherein the chitosan sol forms a solid gel when the temperature is increased to 30-40° C.

2. The method of claim 1, wherein the concentration of the amphiphilically modified chitosan solution is 0.1-3% (w/v).

3. The method of claim 1, wherein the chitosan solution is prepared from 95% deacetylated chitosan powder having a molecular weight of 50 kDa-250 kDa.

4. The method of claim 1, wherein the chitosan solution is hydrophilically modified by haloacetic acid and the haloacetic acid is chloroacetic acid, dichloroacetic acid, trichloroacetic acid, bromoacetic acid, dibromoacetic acid or bromochloroacetic acid.

5. The method of claim 1, wherein the chitosan solution is hydrophobically modified by 2-12 carbons long-chain anhydride and the anhydride is acetic anhydride or hexanoyl anhydride.

6. The method of claim 1, wherein the glaucoma drug is Latanoprost or Timolol maleate.

7. The method of claim 1, wherein the preservative is benzalkonium chloride.

8. The method of claim 1, wherein the basic structure stabilizer is sodium β-glycerophosphate, genipin, sodium bicarbonate, or any combination thereof.

9. The method of claim 1, after the step of forming chitosan gel, further comprises a step of radiating γ-rays at the gel at a dose of 3-10 KGy.

10. A thermosensitive injectable glaucoma drug carrier gel, comprising:
a polymer matrix comprising amphiphilically modified chitosan;
an additive dispersed in the matrix, wherein the additive contains a water/fat soluble glaucoma drug, a preservative, a solvent selected from glycerol, dimethyl sulfoxide (DMSO), ethanol or ethylene glycol, or any combination thereof, and a basic structural stabilizer; and
water.

11. The thermosensitive injectable glaucoma drug carrier gel of claim 10, which is prepared by the method of claim 1.

12. The thermosensitive injectable glaucoma drug carrier gel of claim 10, which is prepared in jelly form or toothpaste form.

13. The thermosensitive injectable glaucoma drug carrier gel of claim 10, which comprises no magnetic-sensitive nanocapsule.
